(19) United States
(12) Patent Application Publication
(10) Pub. No.: US 2011/0085986 A1
(43) Pub. Date: Apr. 14, 2011

(54) Diagnosis and treatment of epithelial cancers using labeled/conjugated progastrin peptides

(76) Inventor: Pomila Singh, Houston, TX (US)

(21) Appl. No.: 12/925,086

(22) Filed: Oct. 13, 2010

Related U.S. Application Data
(60) Provisional application No. 61/278,869, filed on Oct. 13, 2009.

(51) Int. Cl. A61K 49/00; C07K 14/435; A61K 38/17; A61P 35/00

(52) U.S. Cl. 424/9.6; 530/324; 424/9.1; 514/21.3; 514/1.1

(57) ABSTRACT

The current invention provides Progastrin peptides that specifically bind Annexin A2 overexpressed by epithelial cancers. The invention includes isolated homing Progastrin peptides conjugated to an imaging agent and methods of using the same for the diagnosis of epithelial cancers. Also encompassed are Progastrin peptides conjugated to cytotoxic agents such as Camptothecin, Doxorubicin, Paclitaxel and derivatives thereof, and methods of treating epithelial cancer using the same.

### CROSS-REFERENCES TO RELATED APPLICATION

This nonprovisional application claims benefit of priority under 35 U.S.C. §119(e) of provisional U.S. Ser. No. 61/278,869, filed Oct. 13, 2009, now abandoned, the entirety of which is hereby incorporated by reference.

### FEDERAL FUNDING

The invention was supported, in whole or in part, by Grant No. RO1CA097959 from the National Institutes of Health. The Government may have certain rights in the invention.

### BACKGROUND OF THE INVENTION

1. Field of the Invention

The present invention relates generally to the fields of detection, diagnosis, molecular medicine, drug delivery and, more specifically, to diagnosis and treatment of epithelial cancers using labeled and/or conjugated progastrin peptides.

2. Description of the Related Art

A major hurdle to advances in treating cancer is the relative lack of agents that can selectively target the cancer while sparing normal tissue. For example, radiation therapy and surgery, which generally are localized treatments, can cause substantial damage to normal tissue in the treatment field, resulting in scarring and loss of normal tissue. Chemotherapy, in comparison, which generally is administered systemically, can cause substantial damage to organs such as the bone marrow, mucosae, skin and small intestine, which undergo rapid cell turnover and continuous cell division. As a result, undesirable side effects such as nausea, loss of hair and drop in blood cell count often occur when a cancer patient is treated intravenously with a chemotherapeutic drug. Such undesirable side effects can limit the amount of a drug that can be safely administered, thereby hampering survival rate and impacting the quality of patient life. Thus, delivering drugs specifically to tumors, while minimizing exposure to normal tissues is an essential requirement for developing drug delivery systems either for diagnosis or treatment.

The ideal approach to tumor targeting is to capitalize on a molecule that is uniquely expressed on the surface of cancer cells but never on normal tissues—a situation that rarely exists. Realistic scenarios usually involve selecting a molecular target that is over-expressed on tumor cells but is expressed at low levels in other organs. Targeting tumor specific/over-expressing receptors thus offers the possibility of minimizing non-selective toxic effects.

In the past three decades several tumor specific/over-expressing receptors were identified on hematological and epithelial cancers. The targeting component of the diagnostic/therapeutic conjugate is typically an antibody against ‘tumor specific’ receptors. Therapeutic components used for conjugating to the targeting moieties (such as antibodies) have included toxins, cytotoxic chemicals, and radionuclides. Antibodies/antibody fragments conjugated to therapeutic moieties (immuno-conjugates) have been used relatively successfully against hematological malignancies. For example, antigens such as CD22, expressed by white blood cells, have been successfully used for targeting leukemia and lymphomas with immunoconjugates (such as anti-CD22 antibody conjugated to pseudomonas aeruginosa exotoxin A, PE38). However, treating solid tumors with immunoconjugates has not been successful (18). For example anti-HER2/NEUdsFv antibodies genetically fused to PE38 failed to deliver toxicity and lacked clinical efficacy in trials against breast cancers. Other antigens on solid tumors (such as CEA; a 55 kDa breast cancer antigen; CD56 for small cell lung cancers; OVB3 for ovarian cancers) targeted by immunoconjugates have not been clinically successful and caused either hepatic or central nervous system toxicities.

Antibodies are increasingly replaced with targeting ligands such as cytokines or peptides for diagnostic and therapeutic purposes. Ligand (cytokines/growth factors/peptide hormones/peptide mimetics) based therapeutics require that the ligand be internalized after binding to its cognate receptor to exert its cytotoxic effects (19). Internalization of ligand-based therapeutic agents is mediated by the natural interaction between the ligand with its cognate receptor, and overrides multiple drug resistance encountered in cancer cells.

Several cytotoxic-peptide ligands that specifically target membrane receptors on tumor cells are in development. Peptide/receptor molecules, used for diagnostic/therapeutic purposes, have significant limitations. Many of the growth factors/peptide hormone receptors (R), currently being used for targeting (EGF-R, LHRH-R, somatostatin (SST-R), etc) are also expressed by normal cells and play an important role in the normal biology of many organs. There is thus the potential for collateral damage to normal tissues. In some cases the normal receptor is either mutated or down-regulated in cancer cells. For example, CCK2R (normally expressed on epithelial and smooth muscle cells in the gut) was proposed as a target. A high affinity ligand, heptagastrin, conjugated to a cytotoxic agent effectively reduced the growth of mutant NIH/3T3 cells over-expressing CCK2R, in vitro and in vivo (19). However this strategy was not pursued after it became evident that wild type CCK2R are not expressed to a significant extent on cancer cells; efforts to target this receptor with vaccines failed in clinical trials. Similarly growth factor receptors are also expressed on liver cells and neuronal cells, resulting in co-lateral toxicities to the liver/central nervous system. Peptide hormone receptors, expressed in a restricted manner, may be more appropriate targets. LHRH receptors (mainly expressed on pituitary cells) are apparently also over-expressed by breast, ovarian and prostate cancers. LHRH peptides conjugated to either apoptotic agents (camptothecin) or labeled with tritium (to deliver targeted radiation) were reported to significantly reduce tumor size of xenografts (20). While the tumor kill appeared to be rapid, it was incomplete and reached a plateau; the tumor size started increasing after 70 h, suggesting that stem cells and their rapidly dividing progeny, perhaps going through epithelial-mesenchymal-transition (EMT) at the leading edges of the tumors, were not targeted. Similarly, a synthetic somatostatin (SST) analog (octreotide), with improved metabolic stability, conjugated to paclitaxel (Taxol), retains biological activity, and is endocytosed via the SST-R (21). The endocytosed Taxol caused apoptosis of neuro-endocrine cancer cells. However, SST receptors are only present on hormone secreting tumors; targeting SST-R is not applicable to epithelial cancers. Role of many of these receptors in tumorigenic potential of cancer cells remains unknown; thus targeting cancer cells positive for receptors like LHRH/SST-R is not expected to be completely effective, and will likely lead to relapse of the cancer disease.

It is necessary to target membrane receptors for ligands which are essential to the tumorigenic potential of the epithelial cancer cells, and which preferably target both the stem cells and the rapidly proliferating cells at the leading edges of the tumors (which are likely to be at a higher risk for undergoing EMT and metastasis). Cancer cells at the leading edges of the tumors are likely to be present in the circulation, giving rise to metastatic lesions. Novel peptide ligands, identified from phage-displayed libraries, likely bind glycosylated proteins, and are specific to a subset of cancer cells/tumors; their role in the proliferative potential of cancer cells remains a question mark. Thus, diagnostic value of peptides discovered by phage-displayed libraries is likely to be higher than their therapeutic potential.

In order to develop targeting strategies, membrane receptors over-expressed by tumors and several epithelial cancers must be targeted. Several peptide hormone receptors currently being targeted are over-expressed only on specific epithelial cancers, and thus their potential use is limited. For example substance P conjugates target neurokinin type 1 (NK-1) receptors, over-expressed in malignant gliomas (22). Bombesin peptide conjugates target gastrin releasing peptide receptors are apparently over-expressed in prostate cancers (23). The clinical efficacy of these agents, however, remains to be examined.

It is also important to develop conjugated peptides which are not immunogenic, are metabolically stable and can be delivered into the cells with high efficiency. Cytotoxic agents such as truncated diphtheria toxin (DT) are highly immunogenic and hamper further treatment cycles. Modifications of therapeutic conjugates using polyethylene glycol (PEGylation) is a typical method by which immunogenicity can be reduced and also prolong the serum half-life (18). Immunogenicity of most immunoconjugates persist which diminishes efficacy of multiple cycles of treatment. The development of small molecule mimetics, especially of endogenous peptide hormone ligands, allows not only selective tissue targeting but also overcomes immunogenicity issues. Development of novel cytotoxic payloads such as human RNases, small interfering RNAs, that are not immunogenic will help to address this issue further.

For diagnostic purposes, the cytotoxic agents are replaced with diagnostic agents, which are primarily radio-nuclides. The most common radio-labeled peptides used for diagnosing neuroendocrine tumors is indium-111-octreotide. However this is not favorable for detecting small tumor deposits. Gallium-68 conjugated with chelating agent, such as 1,4,7,10-tetraaza cyclodododecane 1,4,7,10 tetra acidic acid (DOTA), to the peptide are ideal compounds for positron emission tomography (PET) imaging (20,23). For example, the peptide conjugate 111In/90Y-DOTAGA-substance P is developed for diagnosing and treating brain tumors (22). The bombesin peptide conjugate, DOTA-PEG-truncated bombesin labeled with 67/68 GA is developed for diagnosis and radionuclide therapy of prostate cancer cells (23).

In summary, there is a recognized need in the art for appropriately harnessing the chemistry of ligand/receptor interaction for developing appropriate, widely applicable tools, for diagnosing and treating epithelial cancers. The present invention fulfills this long-standing need and desire in the art.

### SUMMARY OF THE INVENTION

The present invention is directed to isolated homing progastrin peptides that specifically bind AnnexinA2 expressed by a tumor cell.

The present invention also is directed to isolated homing progastrin peptides that specifically bind AnnexinA2 expressed by a tumor cell, said peptide further comprising an imaging reagent conjugated thereto.

The present invention is directed further to isolated homing progastrin peptides that specifically bind AnnexinA2 expressed by a tumor cell, said peptide further comprising a cytotoxic agent conjugated thereto.

The present invention is directed to a method of detecting epithelial cancer in an individual comprising: administering to the individual an isolated homing Progastrin peptide conjugated to an imaging agent; and determining the location of the imaging in the individual, wherein the location corresponds to the cancer.

The present invention is directed further to a method of treating epithelial cancer in an individual comprising administering to the individual a pharmacologically effective amount of an isolated homing progastrin peptide conjugated to a cytotoxic agent or a pharmaceutical derivative thereof.

The present invention is also directed to compositions useful for diagnosis of epithelial cancer comprising an isolated homing progastrin peptide conjugated to an imaging agent. The present invention is directed further to a composition useful for treating epithelial cancer in an individual comprising an isolated homing progastrin peptide conjugated to a cytotoxic agent.

Other and further objects, features, and advantages will be apparent from the following description of the presently preferred embodiments of the invention, which are given for the purpose of disclosure.

### BRIEF DESCRIPTIONS OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 depicts gastrins which are peptide hormones produced in the antral region of the stomach and post-translationally modified by endopeptidases and amidation on the carboxyl terminus in endocrine cells to convert the precursor progastrin to glycine-extended gastrin (G-Gly) and finally to G-34 or G-17. No processing takes place in serum or epithelial cells including colonocytes. N and C terminal fragments of PG, beyond G34, are indicated.

FIG. 2 shows Relative binding affinity (RBA) of PG, G17 and CCK8 for displacing the binding of either 125I rhPG to ANX-II (i) or 125I-BH-CCK8 to CCK2R (ii) on AR42J cells. Plot points represent mean values of triplicate measurements from a representative experiment. The nM concentrations of each peptide used are presented in a log-scale on the x-axis; the excess unlabeled peptide used for displacing the binding of the radio-labeled ligand is presented in parentheses.

FIG. 3 shows binding and co-IP of rhPG and ANX-II in an in vitro binding assay. Lane 1 is rhPG; lane 2 is ANX-II; lanes 3 and 4 are rhPG and ANX-II; lane 3 is IP with anti-PG (aPG); lane 4 is IP with anti-ANX-II (aANX-II). All the samples were processed for WB with both aPG and ANX-II.

FIG. 4 depicts paraffin embedded human normal colon and Adenoma carcinoma. Sections were deparaffinized, and processed for immunostaining with labeled anti-PG-Ab and anti-ANX-II Ab. Red and green fluorescence represents ANX-II for normal and Ad samples, respectively. The nucleus was stained with DAPI and is seen as blue in the images. Images=40× magnification

FIGS. 5A-5B depict paraffin embedded human normal ovary (FIG. 5A) and tumor (FIG. 5B) tissue. Sections were deparaffinized, and processed for immunostaining with labeled anti-PG-Ab and anti-ANX-II Ab. Green and red fluorescence represents ANX-II for normal and Ad samples, respectively. The nucleus was stained with DAPI and is seen as blue in the images. Images are at 40× magnification.

FIGS. 6A-6E depict the binding of PG with ANXII. FIG. 6A depicts immuno-colocalization of ANXII/PG in IEC cells, incubated with PG. E. IEC-18 cells were seeded, on sterile glass cover slips. Following overnight culture cells were incubated with PG (1 nM) from 0-15 min, and washed 3× with chilled PBS. Cells were fixed in acetone:methanol for 20 min at −20° C., and incubated with anti-PG or anti-ANXII-Abs, followed by fluorescence labeling with second Abs. Images were captured by confocal microscopy at 60×. Insets represent computer assisted enlarged images. Arrows depict co-localization on membranes. FIG. 6B depicts FACScan of IEC-18 cells demonstrating cell surface staining of ANXII. Cells in culture were detached with liberase to make single cell suspensions and incubated with either non-immune IgG or anti-ANXII-Ab for 2 hours at room temperature, washed with chilled PBS followed by incubation with FITC-second-Ab for 1 hours at room temperature. FIG. 6C depicts IEC-18 cells in culture treated with PG for 0-15 min except for Lamp1 (treated for 0-30 min) at 37° C. and washed with chilled PBS 3×. Cells were processed for pull down with anti-PG-Ab and processed for WB analysis for either EEA1/ANXII/PG/Lamp1. FIG. 6D shows Confocal microscopy (60×) of PG and EEA1 in cells treated with PG for 15 minutes. The inset portion of the co-localization staining is also shown. FIG. 6E shows IEC cells, treated with either control or ANX-II specific siRNA for 24 hours, were stimulated with PG for 10 minutes and imaged (n=3). Cells treated with ANX-II siRNA were down-regulated for ANX-II expression.

FIG. 7 depicts IEC-18 cells seeded on sterile glass cover slips and cultured overnight. Cells were washed with PBS and incubated for indicated time period with 300 pmole of FITC-PG26 at 37° C. Cells were then washed with chilled 3×PBS and fixed in acetone:methanol for 20 minutes at −20° C. To stain ANXII, cells were incubated with monoclonal antibody, followed by anti-mouse Ab coupled to Alexa-Fluor-598. The cells were stained with DAPI to stain the nucleus and mounted to get images with a fluorescence microscope.

FIGS. 8A-E depict 10-100 μg of FITC-PG-26 peptide was dissolved in 100 μl of saline was used to detect the tumor in nude mice. The images were captured in live mice after 1-60 minutes, with injection on the tumor site (FIG. 8A) or through tail vein (FIGS. 8B-D). In FIG. 8B, the localization of tumor cells are marked with arrows detected after 5 minutes post tail vein injection. As seen in FIG. 8C, the FITC-PG26 was persistent on the tumor after 60 min post injection. In FIG. 8D, the arrow head shows the metastasized tumor cells in liver and the primary site of the tumor is shown by an arrow. FIG. 8E depicts that Control mice injected with control unrelated peptide, did not show any FITC signal after 30-60 min post injection.

FIG. 9 depicts synthesis scheme for camptothecin analogs with linkers.

FIG. 10 depicts synthesis scheme for doxorubicin analogs with linkers.

FIG. 11 depicts synthesis scheme for paclitaxel analogs with linkers.

FIGS. 12A-12B show the synthetic scheme for generation of PG-drug conjugates.

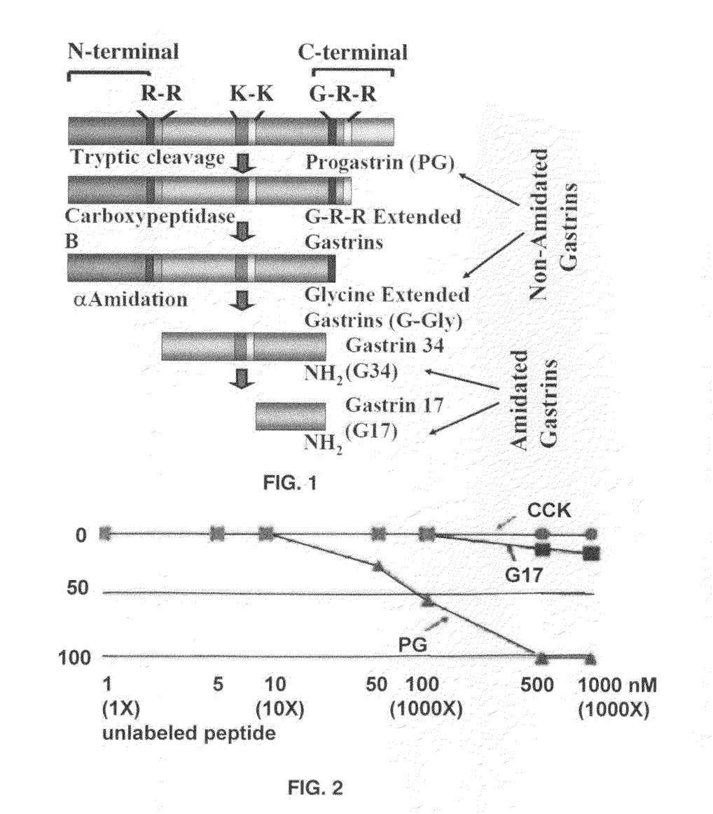

### DETAILED DESCRIPTION OF THE INVENTION

Effectively targeting membrane receptors specifically expressed by many epithelial cancers by utilizing endocytosed bound ligand (conjugated to diagnostic or therapeutic agents), which will have significant health benefits for cancer patients, since epithelial cancers (lung, breast, colon etc) continue to represent the leading causes of cancer death. One such receptor is the membrane associated extracellular Annexin A2 (Annexin A2; p36) molecule. Herein is described the use of extra-cellular Annexin A2, over-expressed on tumor cells, as a target for diagnosis and treatment of epithelial cancer.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques An Intensive Laboratory Course (Ream et al., eds., 1998, Academic Press); PCR (Introduction to Biotechniques Series), 2nd ed. (Newton & Graham eds., 1997, Springer Verlag); Peters and Dalrymple, Fields Virology, 2nd ed., Fields et al. (eds.) (B.N. Raven Press, New York, N.Y.). All publications, patents and patent applications cited herein, whether supra or infra, are hereby incorporated by reference in their entirety.

As used herein the specification, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. Similarly, the word “or” is intended to include “and” unless the context clearly indicates otherwise. “Comprise” means “include.” It is further to be understood that all base sizes or amino acid sizes, and all molecular weight or molecular mass values, given for nucleic acids or polypeptides are approximate, and are provided for description. Although methods and materials similar or equivalents to those described herein can be used in the practice or testing of the present disclosure, suitable methods and materials are described below. In case of conflict, the present specification, including explanations of terms, will control. In addition, the materials, methods, and examples are illustrative only and not intended to be limiting. Furthermore, unless otherwise required by context, singular terms shall include pluralities and plural terms shall include the singular.

The peptides and peptidomimetics of the invention are provided in isolated form. As used herein in reference to a peptide or peptidomimetic of the invention, the term “isolated” means a peptide or peptidomimetic that is in a form that is relatively free from material such as contaminating polypeptides, lipids, nucleic acids and other cellular material that normally is associated with the peptide or peptidomimetic in a cell or that is associated with the peptide or peptidomimetic in a library or in a crude preparation.

The peptides and peptidomimetics of the invention, including the bifunctional, multivalent and homing peptides and peptidomimetics discussed below, can have a variety of lengths. A peptide or peptidomimetic of the invention can have, for example, a relatively short length of less than eight, nine, ten, 12, 15, 20, 25, 30, 35, 40, 45, 50, 60, 70 or 80 residues. A peptide or peptidomimetic of the invention also can be useful in the context of a significantly longer sequence as described further below. As used herein, the term “residue” refers to amino acids or analogs thereof. It is understood that a peptide containing, for example, the amino acid sequence SEQ ID NO: 1 includes the specified amino acids as a contiguous sequence not separated by other amino acids.

The present invention also provides an isolated peptide or peptidomimetic containing an amino acid sequence which is a conservative variant, for example, comprising the sequence of SEQ ID NO 1: QGPWLEEEEEAYGWMDFGRRSAEDEN. As used herein, a “conservative variant” is an amino acid sequence in which a first amino acid is replaced by a second amino acid or amino acid analog having at least one similar biochemical property, which can be, for example, similar size, charge, hydrophobicity or hydrogen-bonding capacity. For example, a first hydrophobic amino acid can be conservatively substituted with a second (non-identical) hydrophobic amino acid such as alanine, valine, leucine, or isoleucine, or an analog thereof. Similarly, a first basic amino acid can be conservatively substituted with a second basic amino acid such as arginine or lysine, or an analog thereof. In the same way, a first acidic amino acid can be conservatively substituted with a second acidic amino acid such as aspartic acid or glutamic acid, or an analog thereof, or an aromatic amino acid such as phenylalanine can be conservatively substituted with a second aromatic amino acid or amino acid analog, for example, tyrosine.

As disclosed herein, a peptide or peptidomimetic of the invention can maintain homing activity in the context of a significantly longer sequence. For example, the 26-mer peptide of SEQ ID NO 1: QGPWLEEEEEAYGWMDFGRRSAEDEN maintained the ability to home when fused to a phage coat protein, confirming that a peptide of the invention can have selective homing activity when embedded in a larger protein sequence. Thus, the invention further provides a chimeric protein containing a peptide or peptidomimetic of the invention, or a homing peptide or peptidomimetic of the invention, fused to a heterologous protein. In one embodiment, the invention provides a chimeric protein containing a homing peptide or peptidomimetic that selectively homes to tumor blood cells or tumor cells and that specifically binds nucleolin fused to a heterologous protein. In one embodiment, the heterologous protein has a therapeutic activity. In a further embodiment, the heterologous protein is an antibody or antigen-binding fragment thereof. In other embodiments, the invention provides a chimeric protein in which a peptide or peptidomimetic containing the amino acid sequence SEQ ID NO: 1, or a conservative variant or peptidomimetic of this sequence, is fused to a heterologous protein. The term “heterologous,” as used herein in reference to a protein fused to a peptide or peptidomimetic of the invention, means a protein derived from a source other than the gene encoding the peptide of the invention or upon which the peptidomimetic is derived. A chimeric protein of the invention can have a variety of lengths, for example, up to 100, 200, 300, 400, 500, 800, 1000 or 2000 residues or more.

The present invention further provides an isolated multivalent peptide or peptidomimetic that includes at least two motifs each independently containing the amino acid sequence SEQ ID NO: 1, or a conservative variant or peptidomimetic thereof. The multivalent peptide or peptidomimetic can have, for example, at least three, at least five or at least ten of such motifs, each independently containing the amino acid sequence SEQ ID NO: 1, or a conservative variant or peptidomimetic thereof. In particular embodiments, the multivalent peptide or peptidomimetic has two, three, four, five, six, seven, eight, nine, ten, fifteen or twenty identical or non-identical motifs of the amino acid sequence SEQ ID NO: 1, or a conservative variant or peptidomimetic thereof. In another embodiment, the multivalent peptide or peptidomimetic contains identical motifs, which consist of the amino acid sequence SEQ ID NO: 1, or a conservative variant or peptidomimetic of this sequence. In a further embodiment, the multivalent peptide or peptidomimetic contains contiguous motifs, which can be identical or non-identical.

Thus, the invention provides peptides and peptidomimetics, including bifunctional and multivalent peptides and peptidomimetics, and homing peptides and peptidomimetics as discussed further below. As used herein, the term “peptide” is used broadly to mean peptides, proteins, fragments of proteins and the like. The term “peptidomimetic,” as used herein, means a peptide-like molecule that has the activity of the peptide upon which it is structurally based. Such peptidomimetics include chemically modified peptides, peptide-like molecules containing non-naturally occurring amino acids, and peptoids, and have an activity such as selective homing activity of the peptide upon which the peptidomimetic is derived (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861).

A variety of peptidomimetics are known in the art including, for example, peptide-like molecules which contain a constrained amino acid, a non-peptide component that mimics peptide secondary structure, or an amide bond isostere. A peptidomimetic that contains a constrained, non-naturally occurring amino acid can include, for example, an alpha.-methylated amino acid; α,α-dialkylglycine or α-aminocycloalkane carboxylic acid; an N^{α}—C^{α}. cyclized amino acid; an N^{α}-methylated amino acid; a β- or γ-amino cycloalkane carboxylic acid; an α,β-unsaturated amino acid; a β,β-dimethyl or β-methyl amino acid; a β-substituted-2,3-methano amino acid; an N—C^{δ} or C^{α}—C^{α} cyclized amino acid; a substituted proline or another amino acid mimetic. A peptidomimetic which mimics peptide secondary structure can contain, for example, a nonpeptidic β-turn mimic; γ-turn mimic; mimic of β-sheet structure; or mimic of helical structure, each of which is well known in the art. A peptidomimetic also can be a peptide-like molecule which contains, for example, an amide bond isostere such as a retro-inverso modification; reduced amide bond; methylenethioether or methylene-sulfoxide bond; methylene ether bond; ethylene bond; thioamide bond; trans-olefin or fluoroolefin bond; 1,5-disubstituted tetrazole ring; ketomethylene or fluoroketomethylene bond or another amide isostere. One skilled in the art understands that these and other peptidomimetics are encompassed within the meaning of the term “peptidomimetic” as used herein.

Methods for identifying a peptidomimetic are well known in the art and include, for example, the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). This structural depository is continually updated as new crystal structures are determined and can be screened for compounds having suitable shapes, for example, the same shape as a peptide of the invention, as well as potential geometrical and chemical complementarity to a target molecule. Where no crystal structure of a peptide of the invention is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of a peptide of the invention, for example, with activity in selectively homing to tumor blood vessels and tumor cells.

Active fragments of the homing peptide disclosed herein as SEQ ID NO: 1 also can be useful in the conjugates and methods of the invention. As used herein in reference to a peptide sequence such as SEQ ID NO: 1, the term “active fragment” means a fragment that has substantially the amino acid sequence of a portion of the 26-amino acid peptide SEQ ID NO: 1 and that retains substantially the selective homing activity of the parent peptide. Selective homing activity can be assayed by routine methods, as described in the Examples below. In one embodiment, an active fragment contains the amino acid sequence of a portion of SEQ ID NO: 1. Such an active fragment can have, for example, the amino acid sequence of at least 10, 12, 15, 18, 20, 22 or 25 contiguous residues of SEQ ID NO: 1.

The present invention also provides a method of imaging tumors and tumor vasculature in a subject by administering to the subject a conjugate containing a detectable label linked to a homing molecule that selectively homes to tumor blood vessels and tumor cells and that specifically binds nucleolin; and detecting the conjugate, thereby imaging tumors and tumor vasculature. A homing molecule useful in an imaging method of the invention can be, for example, a homing peptide or peptidomimetic such as a homing peptide or peptidomimetic that contains the amino acid sequence SEQ ID NO: 1 or a conservative variant or peptidomimetic of this sequence. Any of a variety of detectable labels are useful in the imaging methods of the invention, including fluorescent labels and radionuclides such as indium-111, technetium-99, carbon-11, carbon-13 and Gallium 67/68, which can be linked to the peptide of interest with chemical chelating agents, such as DOTA.

The methods of the invention for imaging tumors and tumor vasculature can be useful for detecting the presence of blood vessels associated with a variety of tumors. Following administration of a conjugate of the invention containing a detectable label, tumor blood vessels are visualized. If the image is positive for the presence of such tumor vessels, the tumor can be evaluated for size and quantity of vascular infiltration. These results provide valuable information to the clinician with regard to the stage of development of the cancer and the presence or probability of metastasis.

In a method of imaging tumors and tumor vasculature, the conjugate administered contains a detectable label that allows detection or visualization of tumor blood vessels and tumor cells, for example, of leukemias or breast cancers. For in vivo diagnostic imaging of such cancers, a homing molecule is linked to a detectable label that, upon administration to the subject, is detectable external to the subject. Such a detectable label can be, for example, a gamma ray emitting radionuclide such as indium-113, indium-115, technetium-99 or Gallium 67/68; following administration to a subject, the conjugate can be visualized using a solid scintillation detector, such as PET scan.

The present invention also provides a method of reducing the number of tumor blood vessels in a subject by administering to the subject a conjugate which contains a cytotoxic agent linked to a homing molecule that selectively homes to tumor blood vessels and tumor cells and that specifically binds nucleolin, thereby reducing the number of tumor blood vessels in the subject. The peptide or peptidomimetic portion of the conjugate can have, for example, a length of at most 200 residues, or a length of at most 50 residues. In one embodiment, a method of the invention is practiced with a conjugate containing a homing peptide or peptidomimetic. In a further embodiment, a method of the invention is practiced with a conjugate containing a homing peptide or peptidomimetic that includes the amino acid sequence SEQ ID NO: 1, or a conservative variant or peptidomimetic of this sequence. Any of the therapeutic moieties described above, such as anti-angiogenic agents, cytotoxic agents and cytotoxic agents that target a DNA-associated process, as well as additional moieties disclosed herein or known in the art, can be used to reduce the number of tumor blood vessels according to a method of the invention.

Further provided herein is a method of treating cancer in a subject by administering to the subject a conjugate which contains a therapeutic moiety linked to a homing molecule that selectively homes to tumor blood vessels and tumor cells and that specifically binds Anx2. In particular embodiments, the peptide or peptidomimetic portion of the conjugate has a length of at most 200 residues, or a length of at most 50 residues. In other embodiments, a method of the invention is practiced with a conjugate containing a homing peptide or peptidomimetic such as a homing peptide or peptidomimetic that includes the amino acid sequence SEQ ID NO: 1, or a conservative variant or peptidomimetic of this sequence. It is understood that, in a method of the invention for treating cancer in a subject, any of a variety of therapeutic moieties can be useful, including but not limited to, cytotoxic agents; and cyclophosphamide, melphalan, mitomycin C, bizelesin, cisplatin, doxorubicin, etoposide, mitoxantrone, SN-38, Et-743, actinomycin D, bleomycin, TLK286 and other cytotoxic agents that target a DNA-associated process.

It is understood that a variety of routes of administration are useful in the methods of the invention. Such routes encompass systemic and local administration and include, without limitation, oral administration, intravenous injection, intraperitoneal injection, intramuscular injection, subcutaneous injection, transdermal diffusion or electrophoresis, local injection; extended release delivery devices, including locally implanted extended release devices such as bioerodible or reservoir-based implants.

A therapeutic moiety useful in a conjugate of the invention can be, for example, a cytotoxic agent. As used herein, the term “cytotoxic agent” refers to any molecule that results in cell death by any mechanism. Exemplary cytotoxic agents useful in a conjugate of the invention encompass, without limitation, taxanes such as docetaxel; anthracyclins such as doxorubicin; alkylating agents; vinca alkaloids; anti-metabolites; platinum agents such as cisplatin or carboplatin; steroids such as methotrexate; antibiotics such as adriamycin; antimicrobial peptides, described herein below; and other cancer chemotherapeutic agents, which are chemical agents that inhibit the proliferation, growth, life-span or metastatic activity of cancer cells.

Taxanes are cytotoxic agents useful in a conjugate of the invention. Useful taxanes include, without limitation, docetaxel (Taxotere; Aventis Pharmaceuticals, Inc.; Parsippany, N.J.) and paclitaxel (Taxol; Bristol-Myers Squibb; Princeton, N.J.). See, for example, Chan et al., J. Clin. Oncol. 17:2341-2354 (1999), and Paridaens et al., J. Clin. Oncol. 18:724 (2000).

A cytotoxic agent useful in a conjugate of the invention also can be an anthracyclin such as doxorubicin, idarubicin or daunorubicin. Doxorubicin is a commonly used cancer chemotherapeutic agent (Stewart and Ratain, In: “Cancer: Principles and practice of oncology” 5th ed., chap. 19 (eds. DeVita, Jr., et al.; J. P. Lippincott 1997); Harris et al., In “Cancer: Principles and practice of oncology,” supra, 1997). In addition, doxorubicin has anti-angiogenic activity, which can contribute to its effectiveness in treating cancer (Folkman, supra, 1997; Steiner, In “Angiogenesis: Key principles-Science, technology and medicine,” pp. 449-454 (eds. Steiner et al.; Birkhauser Verlag, 1992)).

An alkylating agent such as melphalan or chlorambucil also can be a cytotoxic agent useful in a conjugate of the invention. Similarly, vinca alkaloids such as vindesine, vinblastine or vinorelbine; or antimetabolites such as 5-fluorouracil, 5-fluorouridine or a derivative thereof are cytotoxic agents that can be linked to a homing molecule in a conjugate of the invention.

Cytotoxic agents useful in the conjugates of the invention also include platinum agents. Such a platinum agent can be, for example, cisplatin or carboplatin as described, for example, in Crown, Seminars in Oncol. 28:28-37 (2001). Other cytotoxic agents useful in a conjugate of the invention include, without limitation, methotrexate, mitomycin-C, adriamycin, ifosfamide and ansamycins.

As used herein, the term “subject” refers to any target of the treatment. Preferably, the subject is a mammal, more preferably, the subject is a human.

In all embodiments of the present invention is an isolated homing Progastrin peptide that specifically bind Annexin A2 expressed by a tumor cell. In some of these embodiments, the peptide has a length of less than 81 residues. In other embodiments, the peptide has a length of less than 50 residues. Further, in some embodiments, the isolated peptide has a length of less than 40 residues. In yet some other embodiments, the isolated peptide has a length of less than 30 residues. In some embodiments of the current invention, the isolated peptide comprises a sequence shown in SEQ ID NO: 1.

In certain embodiments of the present invention is an isolated homing Progastrin peptide that specifically binds Annexin A2 expressed by a tumor cell, said peptide further comprising an imaging reagent conjugated thereto. Further to these embodiments, the imaging agent may be one or more of Fluorescein, Fluorescein isothiocyanate, Rhodamines, Cyanines, boron-dipyrromethenes, Tetrapyrroles, Arylmethines, Oxazines, Oxadiazoles, Pyrenes, Acridines or derivatives or combinations thereof. In some of these embodiments, the imaging reagent is a radionuclide.

In certain embodiments of the present invention there is provided an isolated homing progastrin peptide that specifically binds Annexin A2 expressed by a tumor cell, said peptide further comprising a cytotoxic agent conjugated thereto. In some of these embodiments, the cytotoxic agent comprises one or more of Camptothecin, Doxorubicin, Paclitaxel or derivatives thereof.

In certain embodiments of the present invention there is provided a method of detecting epithelial cancer in an individual comprising: administering to the individual an isolated homing progastrin peptide conjugated to an imaging agent; and determining the location of the imaging in the individual, wherein the location corresponds to the cancer. In some of these embodiments, the isolated peptide comprises a sequence shown in SEQ ID NO: 1. In certain embodiments of the present invention is a method of treating epithelial cancer in an individual comprising administering to the individual a pharmacologically effective amount of an isolated homing progastrin peptide conjugated to a cytotoxic agent or a pharmaceutical derivative thereof. In some embodiments, the isolated peptide comprises a sequence shown in SEQ ID NO: 1.

In certain embodiments of the present invention are compositions useful for diagnosis of epithelial cancer comprising an isolated homing progastrin peptide conjugated to an imaging agent. In certain embodiments of the present invention is a composition useful for treating epithelial cancer in an individual comprising an isolated homing progastrin peptide conjugated to a cytotoxic agent.

The following examples are given for the purpose of illustrating various embodiments of the invention and are not meant to limit the present invention in any fashion.

### Example 1

### Targeting Annexin A2

The Annexin (Anx) family of proteins is characterized by the presence of a conserved core domain and a variable amino terminal ‘tail’ domain responsible for specialized functions (24). Anx A2 is a multi-functional protein, and can bind p11 forming an (Anx2)₂/(p11)₂ heterotetramer. Anx A2 binds several ligands with relatively high affinity including tPA on endothelial cells and progastrins on epithelial cells. Proteomic analysis of epithelial tumors reveals that Anx A2 is elevated many fold in human epithelial cancers (including renal, lung, pancreas, breast, colorectal cancers, etc) (24-27). Anx A2 is up-regulated in gastric epithelial cancers infected with H. pylori and may play a role in gastric carcinogenesis (27). Importantly Anx A2 is not detected on quiescent cells in the liver and elsewhere (24). Anx A2 is especially over-expressed at the leading edges of the tumor (27), suggesting the presence of this membrane receptor on rapidly proliferating tumor cells. Since it is over-expressed by many epithelial cancers, extracellular membrane associated Anx A2 can be used as a novel target for diagnostic and therapeutic purposes.

### Example 2

### Progastrin Peptides for Targeting Anx A2

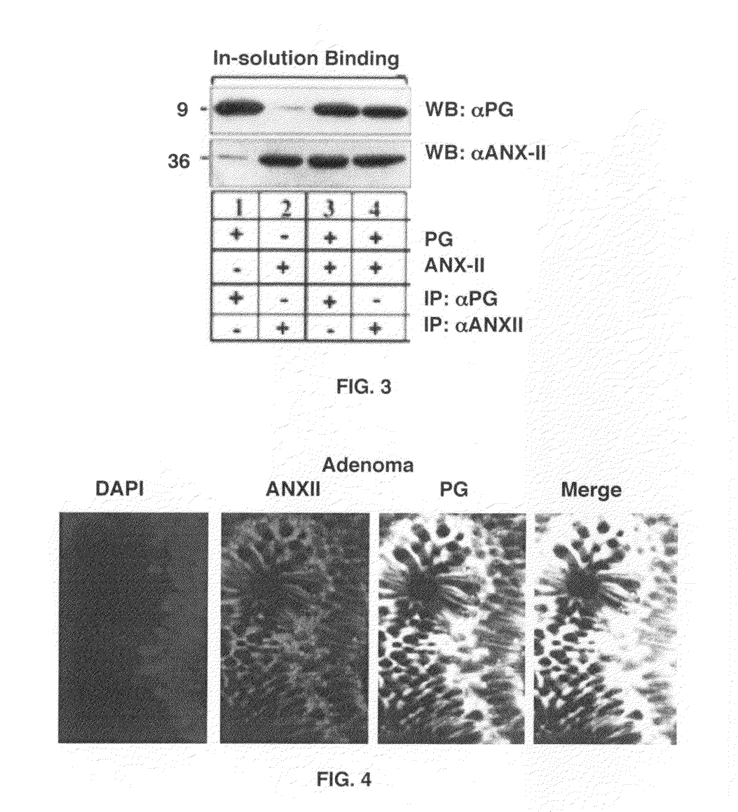

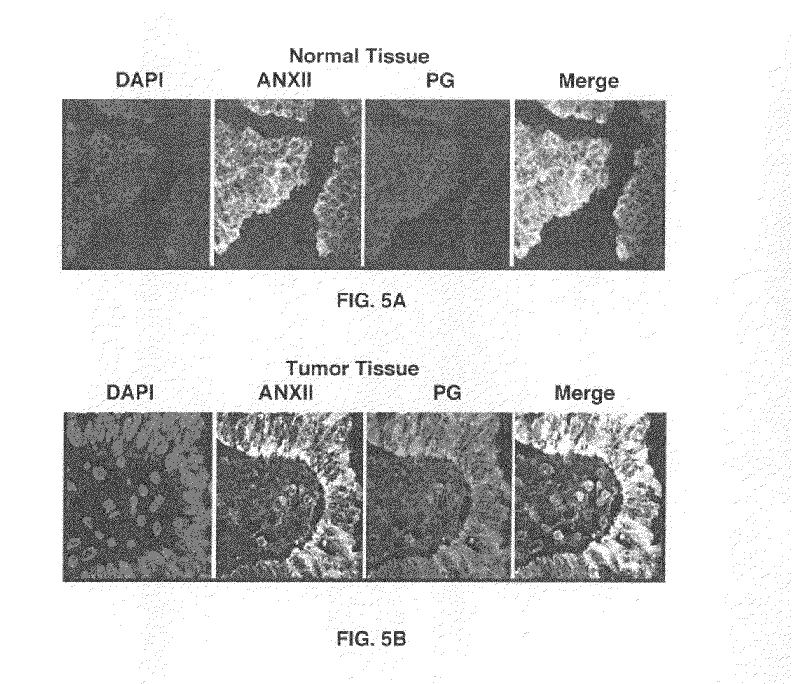

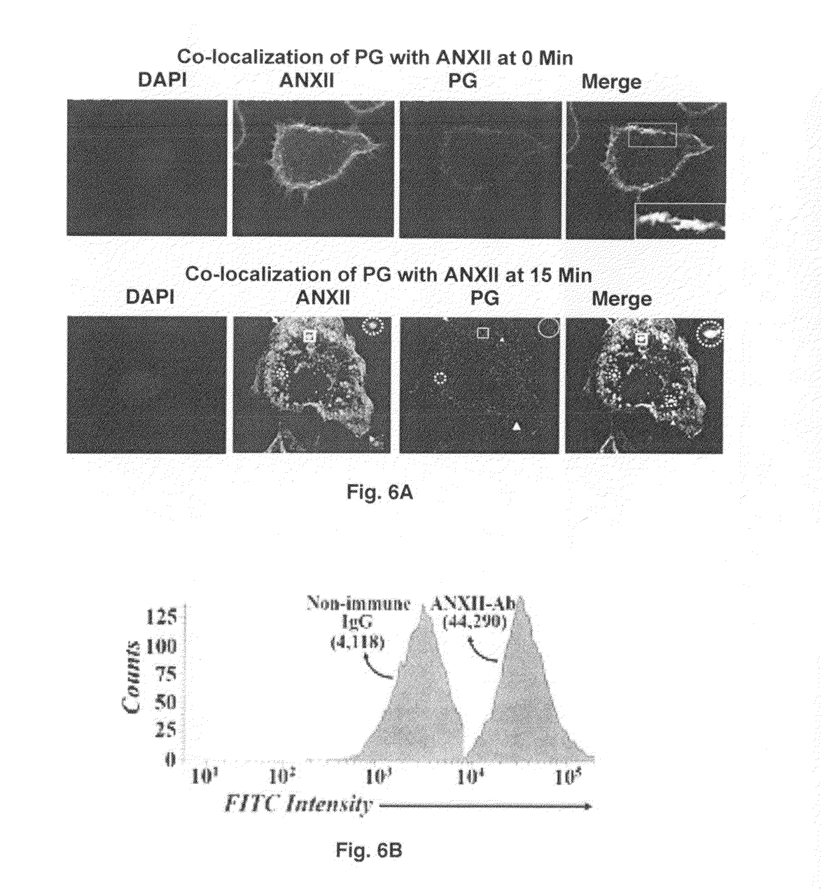

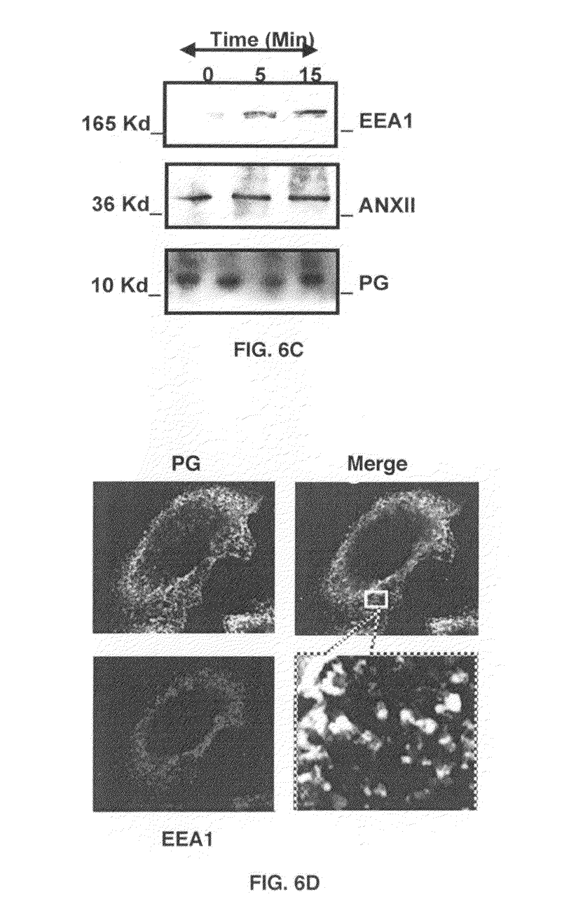

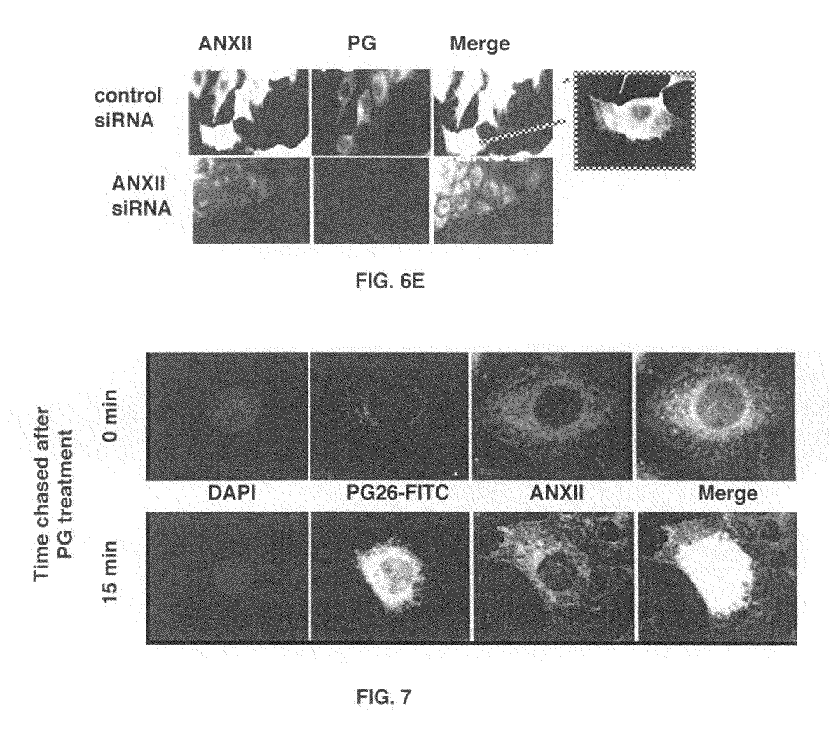

Relative binding affinity (RBA) of gastrins for the 36 kDa Anx A2 protein, prepared from cancer cells/tumor membranes was in the order of PG>G-Gly>G17, with no affinity for cholecystokinin CCK8 (28). While PG peptides demonstrated a high affinity for Anx A2, amidated gastrins (G17, G34) demonstrated a much higher affinity for CCK1R/CCK2R (FIG. 2). Direct binding of Anx A2 and rhPG was further established in an in vitro binding assay (FIG. 3). Functional significance of PG binding to Anx A2 was strongly suggested by the fact that rapidly dividing immortalized cells (IEC, HEK-293) and cancer cell lines (from ovaries/colons), responsive to the growth effects of PG peptides, demonstrated strong co-localization of PG with Anx A2. Adenoma and adenocarcinomas tissue sections (from ovarian and colorectal cancers of patients) were positive for Anx A2 expression, while normal tissues were negative (FIGS. 4, 5A-5B). Immortalized intestinal epithelial cell line (IEC-18) are used, which responds to the growth and anti-apoptotic effects of PG peptides (9), for further investigating the biology of interaction of PG with Anx A2. Intra-cellular translocation of labeled PG was examined in IEC-18 cells. Addition of PG to IEC-18 cells resulted in initial co-localization of PG with Anx A2 on the cellular membranes, followed by rapid intracellular localization of PG/Anx A2 (29) (FIG. 6A). Using fluoroscense assisted sorting with labeled anti-Anx A2-Abs, presence of extracellular Anx A2 was confirmed on IEC-18 cells (FIG. 6B).

Since Anx A2 lacks transmembrane domains, mechanisms mediating translocation of Anx A2/PG from outer cell membranes towards intracellular compartments remains unknown. Anx A2 also binds transmembrane proteins such as CD44, which are over-expressed in cancer cells. Thus the tumor specific expression of CD44-like transmembrane proteins offers an additional level of cancer-specific regulation, allowing intra-cellular translocation of PG-Anx A2 only in tumor cells. The co-localized Anx A2/PG complexes on IEC-18 cells have a punctate appearance (inset in FIG. 6D), suggesting endosomal localization of Anx A2/PG. Co-localization of PG/Anx A2 with the early endosomal marker (EEA1) was examined. Cellular lysates from IEC-18 cells, treated with PG for 0, 5 and 15 min, were pulled down with anti-PG Abs and processed for WB analysis for EEA1, Lamp1 (lysomal marker), Anx A2 and PG (FIG. 6C). Possible in situ co-localization of EEA1 with PG was also examined after 15 min of PG stimulation by confocal microscopy (FIG. 6D). Data in FIGS. 6A-D strongly suggest that Anx A2 and PG translocate into early endosomes within 5 min of PG stimulation, and that PG may increasingly traffic to the degradative pathway (as indicated by a ˜5-fold increase in co-IP of Lamp1 with PG at later time points, FIG. 6C). The role of Anx A2 in intracellular translocation of PG in IEC18 cells was further confirmed by transiently transfecting the cells with either non-specific control siRNA or specific siRNA against Anx A2 transcripts. Down-regulation of Anx A2 expression was confirmed by Western Blot analysis, and cells were plated on glass cover slips and treated with PG. Cells treated with specific siRNA were down-regulated for Anx A2 expression and completely negative for PG binding/uptake (FIG. 6E). Taken together, the data suggests that Anx A2 is required for binding and translocating progastrin peptides to endosomal compartments of Anx A2 expressing cells, by as yet unknown transmembrane mechanisms, which likely include transmembrane protein such as CD44 over-expressed in tumor cells.

### Example 3

### Affinity of Labeled Progastrin (PG) Peptides for Tumors Overexpressing Anx A2

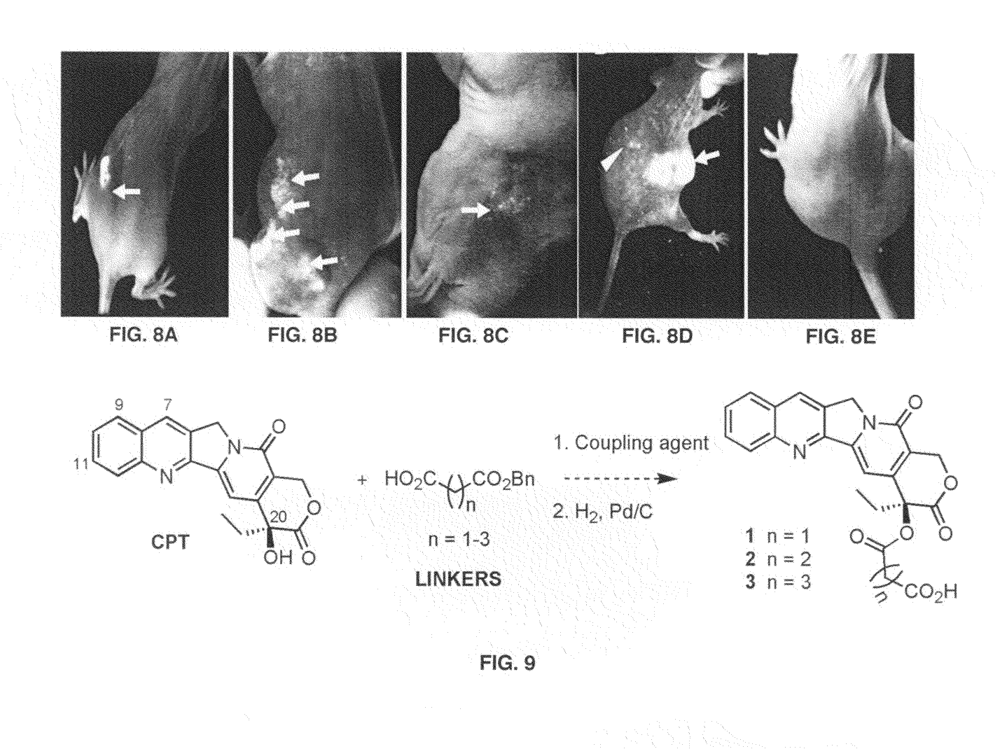

It was critical to demonstrate that labeled PG peptides can home to tumors, in situ, which are over-expressing Anx A2. For data presented in FIGS. 2-4, 5A-5B and 6A-6E, the full length 80 amino acid rhPG was used (SEQ ID NO: 2), which was visualized by staining with fluorescently labeled primary or secondary antibodies. However, in order to examine the feasibility of using PG peptides for diagnostic and treatment purposes, synthesized FITC labeled PG fragments (PG26, SEQ ID NO 1: QGPWLEEEEEAYGWMDFGRRSAEDEN) conjugated with either radio- or fluorescently labeled imaging reagents (for diagnostic purposes), or with cytotoxic payloads (for therapeutic purposes) was studied. PG26 was biologically active. Fluorescein isothiocyanate (FITC) labeled PG26 peptide demonstrated an equivalent binding affinity for Anx A2 as rhPG. Endocytotic internalization of FITC-PG26 in PG responsive immortalized kidney embryonic cell line, HEK-293, was examined. Strong peri-nuclear co-localization of FITC-PG26 with Anx A2 was measured within 15 min of labeling in a chase experiment (FIG. 7). Within 30-60 min FITC-PG26 was apparently degraded suggesting lysosomal degradation of the peptide, confirming the data presented in FIG. 6C. The homing potential of FITC-PG26 to Anx A2 over-expressing tumors, growing as xenografts in nude mice, was confirmed, as shown in FIGS. 8A-8E.

The 26 amino acid PG peptide (PG26), labeled with FITC at the N-terminal end, specifically homed to the site of a colon cancer xenograft, growing either under the skin or growing as a metastatic lesion in the liver. FITC-PG26, injected intra-tumorally (FIG. 8A) retained the fluorescence for >20 mins (without demonstrating any spread of the dye) confirming tumor specific retention of endocytosed FITC-PG26. FITC-PG26 successfully homed to the tumor site after i.v. injection through the tail vein, within 5 min of injection, and was retained in the tumor for ˜60 min (FIG. 8C). Tumor homing was successfully observed in both the sub dermal tumor xenograft and in the metastatic lesion in the liver (FIG. 8D). Even small tumor lesions were detected with FITC-PG26 after 15 min of tail vein injection (FIG. 8B). FITC-control peptide was not detected after 30 min of injecting into the tail vein (FIG. 8E), confirming the specificity of FITC-PG26 peptide for homing to tumors in situ.

PG peptides are not normally present in the circulation, but in patients with hypergastrinemic diseases and/or colorectal cancers, detectable levels of circulating PG/glycine extended gastrin (G-gly) are measured in the circulation. Since PG peptides are endogenous molecules, they are not expected to generate immunogenic responses. Studies demonstrate that there is a rapid turnover of membrane associated Anx A2 (either due to recycling or due to de-novo synthesis), which is expected to overcome competition from endogenous PG peptides (<1 nM), measured in patients with colorectal cancers.

### Example 4

### Design, Synthesis and Pharmacological Evaluation of Cytotoxics-PG Conjugates as Prodrugs for Tumor Specific Delivery/Kill

The anthracyclines, camptothecin analogs and taxol group are three major chemical phenotypes on the market for cancer. The anthracyclines and camptothecins target the DNA topoisomerases while drugs in the taxol group target the microtubules. Although these are effective in killing tumors their cytotoxic effects on the normal cells and tissues lead a variety of side effects. The last two decades has seen a paradigm shift with significant increase in the development of therapies against specific targets by design. Alongside the anticancer drug development field is also focused on testing marketed drugs conjugated to peptides that specifically bind cancer cells for tumor specific delivery.

Based on the unexpected discovery that PG binds Anx A2 leading to the rapid internalization of the PG-Anx A2, it is beneficial to use PG as a carrier to specifically deliver drugs to the tumor. Therefore the following studies are conducted (a) synthesis of camptothecin, doxorubicin and taxol conjugated to the PG delivery peptide (SEQ ID NO 1: QGPWLEEEEEAYGWMDFGRRSAEDEN) (b) optimization of the PG delivery peptide for increased Anx A2 binding and (c) conjugation of the optimal PG peptide to the cytotoxic agents for tumor specific delivery.

### Synthesis of Camptothecin Analogs with Linkers Suitable for PG Peptide Conjugation

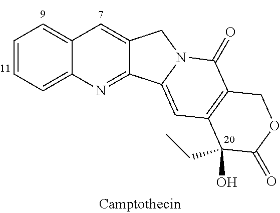

Camptothecin is a pentacyclic quinoline alkaloid that has been the lead compound in several drug development programs for the past fifty years. Structure activity relationship (SAR) of camptothecin suggests the following requirements for cytotoxic activity; (i) the hydroxyl group at position 20 and its absolute S configuration, (ii) the lactone ring (E) and (iii) the planarity of the ring systems, while functionalization of positions 7, 9, 10 and 11 leads to improved activity in vitro and in vivo. The hydroxyl group at position 20 on camptothecin has been used previously to conjugate peptide ligands to explore improved efficacy. Herein, the N-terminus of the PG peptide is conjugated to the hydroxyl group at position 20 through a dicarboxylic acid linker (FIG. 9). The CPT analogs 1-3 are easily coupled to the PG peptide using standard peptide coupling protocols.

### Synthesis of Doxorubicin Analogs with Linkers Suitable for PG Peptide Conjugation

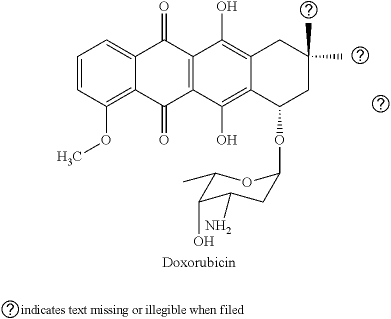

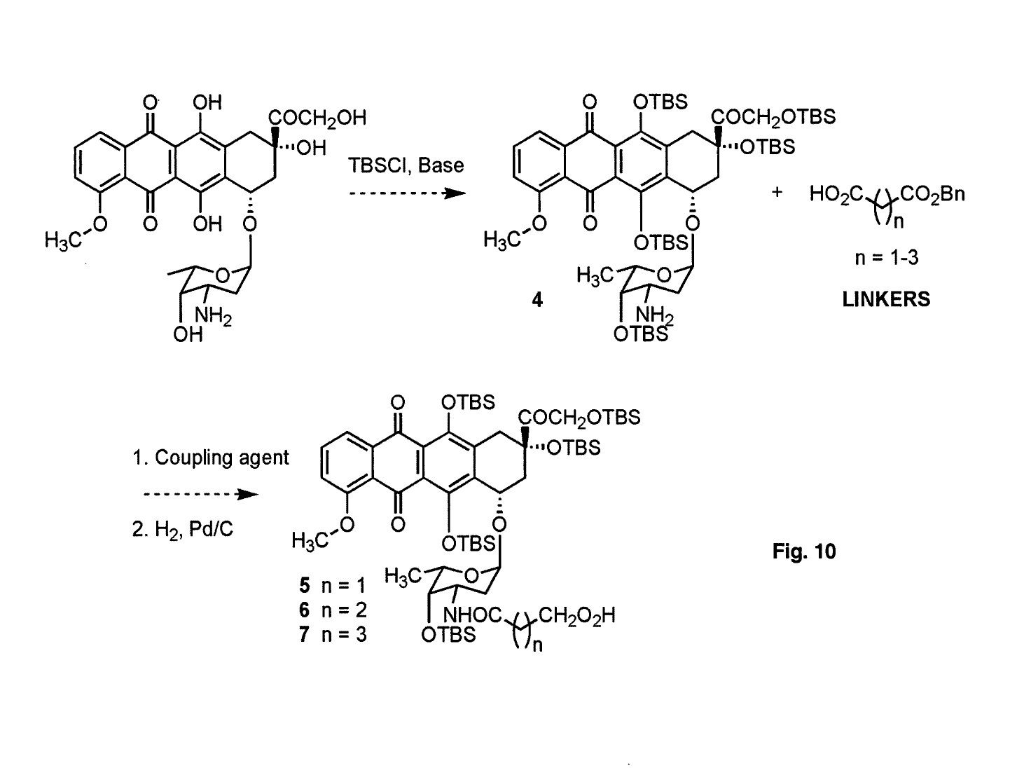

Doxorubicin is a tetracycline that is tethered to a aminohydroxy-tetrahydropyran ring through an ether linkage. It has two phenolic hydroxyl groups, two secondary hydroxyl groups and a primary hydroxyl in addition to a secondary amine. Previous studies have shown that a peptide can be conjugated to the amine without altering the cytotoxic activity. A series of doxorubicin analogs are shown. The oxygen atoms are protected with silyl group which facilitate selective coupling of the linker to the amine. The doxorubicin analogs 5-7 are easily coupled to the PG peptide using standard peptide coupling protocols (FIG. 10).

### Synthesis of Paclitaxel Analogs with Linkers Suitable for PG Peptide Conjugation

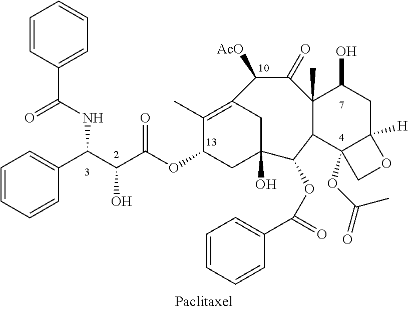

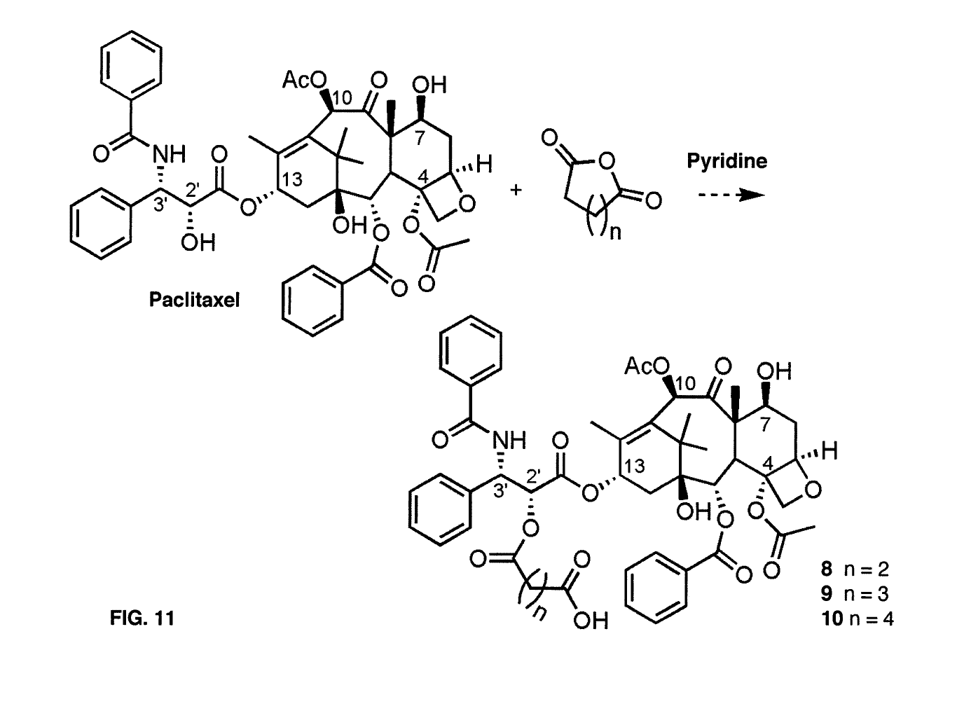

Paclitaxel belongs to the taxol family of antimitotic agents that facilitates tubulin assembly leading to stable aggregates. Paclitaxel contains two hydroxyl groups at C2′ and C7 positions. SAR studies on paclitaxel revealed that (i) an ester group at C13 is critical for its tubulin assembly activity in vitro and cytotoxicity in vivo, (ii) oxidation of C10 to the corresponding ketone dramatically decreased the cytotoxicity while acetylation does not alter the activity and (iii) the esterification of C2′ or C7 resulted in a dramatic loss of the in vitro activity, however, it did not affect the cytotoxicity in vivo, suggesting rapid hydrolysis of the corresponding esters. The hydroxyl at the 2′ position can be selectively functionalized using anhydrides in pyridine to yield monoesters as the major product. Based on this, three paclitaxel analogs 8-10 are generated and readily coupled to the PG peptides using standard coupling protocols (FIG. 11).

### Synthesis of PG-Drug Conjugates

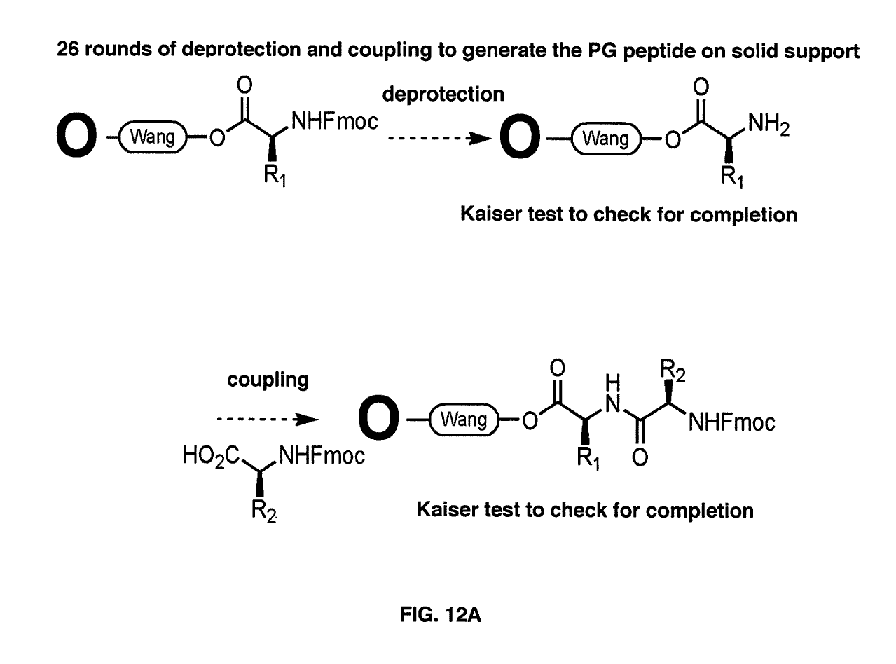

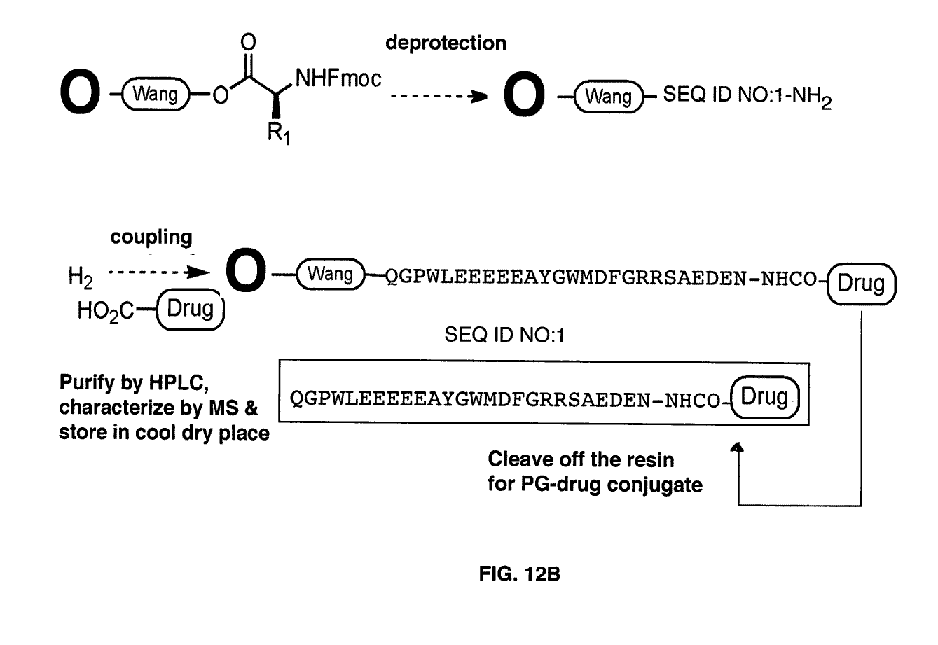

The PG peptide was synthesized (APS peptide synthesizer) on Wang resin using standard Fmoc peptide coupling. In the final step the peptide was divided into nine portions and coupled to each of the nine drug analogs described above. Subsequently the PG-drug conjugates were cleaved off the resin, purified by HPLC and characterized by MS (FIG. 12).

### Example 5

### Optimization of the PG Peptide for Increased Anx2 Binding and Stability

An Ala scan of the PG peptide (SEQ ID NO: 1) was conducted where in each residue was replaced with alanine. Evaluation of this set for binding with Anx A2 allowed identification of critical residues required for binding. Based on this data a second set of truncated peptides of various sizes was generated to identify the minimal unit required for binding. In a subsequent library each position that was not critical was degenerated to identifying the optimal residue at each position required for binding. A second round of oriented peptide library was generated using unnatural amino acids to further optimize each site for increased binding to Anx A2.

### Improving In Vivo Stability and Increase the Half-Life of the Optimized Peptide

The in vivo stability of the optimized peptides was improved by using one or all of the following strategies (i) Methylating critical residues that are susceptible to proteases, for example in SEQ ID NO: 1, amides at residue 4, 5, 14 and 17 are more susceptible than the rest, therefore these amide bond nitrogens are methylated to improve their stability, (ii) reversing the sequence and using D-amino acids, these peptide side chains orient exactly like the side chains of the L-amino acid peptide, while the amide bonds throughout are reversed making them resistant to cellular proteases and peptidases, (iii) use of peptide isosteres such as double bonds or fluorinated double bonds to replace susceptible amide bonds.

### Example 6

### Evaluation of the PG-Drug Conjugates

Binding affinities of the peptides/PG-drug conjugates with Anx A2 was measured using isothermal titration caloriemetry (ITC) and surface plasmon resonance (SPR). ITC will provide the association constants (Kₐ) while SPR provided the kinetic on and off constants (Kₒₙ and K_{off}). The peptides/PG-drug conjugates that provide the highest Kₐ and the best K_{off}, which are indicators of binding affinities and residence times on the receptor respectively, were chosen for further studies. The biological activity of the PG-conjugates was confirmed in a binding assay (as shown in FIG. 2), and by co-localization of the conjugate with Anx A2, using confocal microscopy in IEC, HEK cells (as described in FIGS. 6A-6E). The biologically active conjugate was then be used for treating tumor bearing nude mice, as described in FIGS. 8A-8E, and inhibitory effects on tumor growth examined.

The following references were cited herein:
1. Rengifo-Cam W, Singh P. Curr Pharm Des 2004; 10:2345-2358.
2. Caplin et al., Br J Surg 2000; 87: 1035-1040.
3. Koh et al., Cancer Research 2004; 64: 196-201.
4. Rehfeld J F, Bardram L, Hilsted L. Cancer Res 1989; 49: 2840-2843.
5. van Solinge et al., Cancer Res 1993; 53: 1823-1828.
6. Singh et al., Cancer Res 1996; 56: 4111-4115.
7. Ciccotosto et al., Gastroenterology 1995; 109:1142-1153.
8. Siddheshwar R K, Gray J C, Kelly S B. Gut 2001; 48:47-52.
9. Singh et al., Am J Physiol Gastrointest Liver Physiol 2003; 284: G328-G339.
10. et al., Gastroenterology. 2007; 133 (5): 1554-68.
11. Wu et al., Am J Physiol Gastrointest Liver Physiol 2003; 285: G1097-G1110.
12. Umar et al., Oncogene. 2 Jun. 2008; doi: 10.1038/onc.2008.169
13. Wang et al., J Clin Invest 1996; 98:1918-1929.
14. Cobb et al., Cancer 2004; 1311-1323.
15. Singh et al., Am J Physiol Gastrointest Liver Physiol 2000; 278: G390-G399.
16. Singh et al., Gastroenterology 2000; 119: 162-171.
17. Singh P and Singh G. Gastroenterology 132 (Suppl 2), A-400: A-69-70 2007.
18. Brumlik et al., Expert Opin Drug Deliv. 2008; 5(1): 87-103.
19. Czerwinski et al., Proc Natl Acad Sci USA. 1998; 95(20): 11520-11525.
20. Dharap et al., Proc Natl Acad Sci USA, 2005; 102 (36): 12962-67.
21. Huang C M, Wu Y T, Chen S T. Chem. Biol. 2000; 7(7): 453-461.
22. Kneifel et al., Eur J Nucl Med Mil Imaging. 2007; 34(9): 1388-95
23. Zhang et al., Eur J Nucl Med Mol. Imaging. 2007; 34(8): 1198-1208.
24. Singh P. Role of Annexin-II in GI cancers: interaction with progastrins. Cancer Lett. 2007; 252(1): 19-35.
25. Emoto et al., Cancer. 2001; 92: 1419-1426.
26. Sharma M C, Sharma M. Curr Pharm Des. 2007; 13: 3568-3575.
27. Ortiz-Zapater et al., Am J. Pathol. 2007; 170 (5): 1573-1584.
28. Singh P, Wu H, Clark C, Owlia A. Oncogene. 2007; 26: 425-440.
29. Sarkar S, Singh P. Gastroenterology. Suppl. 1. 2008; 134: A246.

Any patents or publications mentioned in this specification are indicative of the levels of those skilled in the art to which the invention pertains. These patents and publications are herein incorporated by reference to the same extent as if each individual publication was specifically and individually incorporated by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. An isolated homing progastrin peptide that specifically binds Annexin A2 expressed by a tumor cell.

2. The isolated peptide of claim 1, which has a length of less than 81 residues.

3. The isolated peptide of claim 1, which has a length of less than 50 residues.

4. The isolated peptide of claim 1, which has a length of less than 40 residues.

5. The isolated peptide of claim 1, which has a length of less than 30 residues.

6. The isolated homing peptide of claim 1, comprising the amino acid sequence shown in SEQ ID NO: 1.

7. The isolated homing peptide of claim 1, further comprising an imaging reagent conjugated thereto.

8. The isolated homing peptide of claim 7, wherein said imaging reagent is selected from the group consisting of Fluorescein, Fluorescein isothiocyanate, Rhodamines, Cyanines, boron-dipyrromethenes, Tetrapyrroles, Arylmethines, Oxazines, Oxadiazoles, Pyrenes, Acridines, derivatives and combinations thereof.

9. The isolated homing peptide of claim 8, wherein said imaging reagent is Fluorescein isothiocyanate (FITC).

10. The isolated homing peptide of claim 7, wherein said imaging reagent is a radionuclide selected from the group consisting of Gallium 67/68 or Indium 111.

11. The isolated homing peptide of claim 1 further comprising a cytotoxic agent conjugated thereto.

12. The isolated homing peptide of claim 11, wherein said cytotoxic agent is selected from the group consisting of Camptothecin, Doxorubicin, Paclitaxel and derivatives thereof.

13. The isolated homing peptide of claim 1, wherein said tumor is epithelial tumor.

14. A method of detecting cancer in an individual comprising:
administering to the individual the isolated homing peptide of claim 1 conjugated to an imaging agent; and
determining the location of said imaging in the individual, wherein the location corresponds to the cancer.

15. The method of claim 14, wherein said imaging reagent comprises a radionuclide or one or more of Fluorescein, Fluorescein isothiocyanate, Rhodamines, Cyanines, boron-dipyrromethenes, Tetrapyrroles, Arylmethines, Oxazines, Oxadiazoles, Pyrenes, Acridines or derivatives thereof.

16. The method of claim 14 wherein said cancer is epithelial cancer.

17. A method of treating cancer in an individual comprising:
administering to the individual a pharmacologically effective amount of the isolated homing peptide of claim 1 conjugated to a cytotoxic agent or a pharmaceutical derivative thereof.

18. The method of claim 17, wherein said cytotoxic agent comprises one or more of camptothecin, doxorubicin, paclitaxel or derivatives thereof.

19. The method of claim 17 wherein said cancer is epithelial cancer.

20. An isolated homing progastrin peptide of sequence shown in SEQ ID NO: 1, that specifically binds annexin2 expressed by a tumor cell.

21. A composition useful for diagnosis of epithelial cancer comprising:
an isolated homing progastrin peptide of sequence shown in SEQ ID NO: 1 conjugated to an imaging agent.

22. A composition useful for treating epithelial cancer in an individual comprising:
an isolated homing progastrin peptide of sequence shown in SEQ ID NO: 1 conjugated to a cytotoxic agent.

23. A method of detecting epithelial cancer in an individual comprising:
administering to the individual an isolated homing progastrin peptide of sequence shown in SEQ ID NO: 1 conjugated to an imaging agent; and
determining the location of said imaging in the individual, wherein the location corresponds to the cancer.

24. A method of treating epithelial cancer in an individual comprising:
administering to the individual a pharmacologically effective amount of an isolated homing progastrin peptide of sequence shown in SEQ ID NO: 1 conjugated to a cytotoxic agent or a pharmaceutical derivative thereof.

25. An isolated homing progastrin peptide that specifically binds annexin2 expressed by a tumor cell, wherein said peptide has a sequence 95% identical to the amino acid sequence shown in SEQ ID NO: 1.

26. An isolated homing progastrin peptide that specifically binds annexin2 expressed by a tumor cell, wherein said peptide has a sequence 90% identical to the amino acid sequence shown in SEQ ID NO: 1.

27. An isolated homing progastrin peptide that specifically binds annexin2 expressed by a tumor cell, wherein said peptide has a sequence 85% identical to the amino acid sequence shown in SEQ ID NO: 1.

28. The isolated homing peptide of claim 27, further comprising an imaging reagent conjugated thereto.

29. The isolated homing peptide of claim 28, wherein said imaging reagent is selected from the group consisting of fluorescein, fluorescein isothiocyanate, rhodamines, cyanines, boron-dipyrromethenes, tetrapyrroles, arylmethines, oxazines, oxadiazoles, pyrenes, acridines, derivatives and combinations thereof.

30. The isolated homing peptide of claim 28, wherein said imaging reagent is a radionuclide selected from the group consisting of Gallium 67/68 or Indium 111.

31. The isolated homing peptide of claim 27 further comprising a cytotoxic agent conjugated thereto.

32. The isolated homing peptide of claim 31, wherein said cytotoxic agent is selected from the group consisting of camptothecin, doxorubicin, paclitaxel and derivatives thereof.


SEQUENCE LISTING (2 sequences)

SEQ ID NO: 1 - protein, 26 aa, Artificial sequence
  homing progastrin peptide specifically binding annexin2
QGPWLEEEEEAYGWMDFGRRSAEDEN

SEQ ID NO: 2 - protein, 80 aa, Artificial sequence
  recombinant human progastrin
SWKPRSQQPDAPLGTGANRDLELPWLEQQGPASHHRRQLGPQGPPHLVADPSKKQGPWLEEEEEAYGWMDFGRRSAEDEN